(19) United States
(12) Patent Application Publication
(10) Pub. No.: US 2007/0160645 A1
(43) Pub. Date: Jul. 12, 2007

(54) PostConditioning System And Method For The Reduction Of Ischemic-Reperfusion Injury In The Heart And Other Organs

(76) Inventor: Jakob Vinten-Johansen, Grayson, GA (US)

Correspondence Address: NEEDLE & ROSENBERG, P.C., SUITE 1000, 999 PEACHTREE STREET, ATLANTA, GA, 30309-3915, US

(21) Appl. No.: 11/689,992

(22) Filed: Mar. 22, 2007

Related U.S. Application Data
(63) Continuation-in-part of application No. 10/499,052, filed on Jun. 16, 2004.
(63) Continuation-in-part of application No. 10/493,779, filed on Jun. 4, 2004.
(63) Continuation-in-part of application No. PCT/US05/46417, filed on Dec. 20, 2005.
(60) Provisional application No. 60/343,275, filed on Dec. 21, 2001.
(60) Provisional application No. 60/351,203, filed on Oct. 25, 2001.
(60) Provisional application No. 60/638,461, filed on Dec. 22, 2004.

(51) Int. Cl. A61M 29/00

(52) U.S. Cl. 424/423; 604/98.01

(57) ABSTRACT

A postconditioning method and system for preventing injury to an organ or tissue in a subject during or after reperfusion following an ischemic event to the organ or tissue. The system can exemplarily include a catheter configured for insertion therein a vessel of the subject; a means for modulating the flow of fluid within the vessel of the subject; and a control system in communication with the means for modulating the flow of fluid configured to control pulsation of the flow of fluid within the vessel for a desired period of time and at a desired pulsatile frequency.

This application is a continuation-in-part of U.S. patent application Ser. No. 10/499,052, filed Jun. 16, 2004, which is a National Stage Application of International Application No. PCT/US02/41354, filed Dec. 20, 2002, which claims priority from U.S. Provisional Patent Application No. 60/343,275, filed Dec. 21, 2001; and is a continuation-in-part of U.S. patent application Ser. No. 10/493,779, which is a National Stage Application of International Application No. PCT/US02/34158, filed Oct. 25, 2002, which claims priority to U.S. Provisional Patent Application No. 60/351,203, filed Oct. 25, 2001; and is a continuation-in-part of International Application No. PCT/US05/46417, filed Dec. 20, 2005, which claims priority to U.S. Provisional Application No. 60/638,461, filed Dec. 22, 2004, all of the aforementioned applications being incorporated herein by this reference in their entirety.

### FIELD OF THE INVENTION

This invention relates to the treatment of organs and tissues injured by ischemia. More particularly, the present invention relates to preventing or reducing reperfusion injury or perfusion related injury in organs and tissues that have suffered an ischemic event.

### BACKGROUND OF THE INVENTION

Heart disease is the leading cause of premature, permanent disability among American workers, accounting for nearly 20 percent of Social Security disability payments. About 20 million Americans live with the effects of heart disease, and over six million people have heart attacks each year. Every year nearly 50% of patients suffering first-time heart attacks die from myocardial infarctions.

The heart needs a constant and uninterrupted blood supply for normal and continued function. When a patient has a heart attack, the blood flow to part of the heart is stopped, resulting in ischemia. The heart will lose its functional capabilities, and the ischemic part of the heart is in jeopardy of dying, resulting in focal necrosis of the heart tissue. A heart attack can be treated either by percutaneous transluminal coronary angioplasty (PTCA) or by a more invasive procedure, coronary artery bypass graft surgery (CABG). Both procedures can open up a blocked blood vessel (coronary artery) to restore blood supply to the heart muscle, a process called reperfusion. Reperfusion can also be applied to transplanted organs such as, for example, hearts, livers, pancreases, kidneys, and the like.

One well accepted treatment for coronary artery disease, which can occur when blood flow to the heart is restricted due to hardened, plugged up coronary arteries, is PTCA. In the case of an acute myocardial infarction (MI), a coronary artery is typically occluded by a blood clot forming on top of a pre-existing fixed blockage/lesion. Conventionally, PTCA may be performed using the following technique. The physician uses local anesthetic to numb a specific area of the patient's body, usually the upper thigh area where the femoral artery is. A small tube called a sheath is inserted into an artery, such as the femoral artery. A flexible balloon-tipped plastic catheter is inserted through the sheath, advanced to the heart and directed to an area of coronary blood vessel narrowing. The balloon is subsequently inflated to force the blockage against the vessel wall to at least partially reopen the vessel. With the blood flow restored, the balloon catheter is then deflated and removed.

In another exemplified technique that is commonly used in conjunction with balloon angioplasty to treat coronary artery disease, is a catheter-based procedure in which a stent, which is usually an expandable wire mesh tube or scaffolding, is inserted into a diseased artery after the angioplasty reduces the narrowing of the coronary artery to hold open the artery. It is contemplated that the present invention can be an adjunct to any therapies that are used to treat the underlying disease, such as, for example, coronary artery disease, other organs being treated for vessel obstructions, transplants, desired localized control of the flow of fluid in a vessel, and the like.

Timely reperfusion to reduce the duration of ischemia is the definitive treatment to prevent cellular injury and necrosis in an ischemic organ of tissue. Typically reperfusion, after a short episode of myocardial ischemia (up to 15 min), is followed by the rapid restoration of cellular metabolism and function. Even with the successful treatment of occluded vessels, a significant risk of additional tissue injury after reperfusion may still occur.

If the ischemic episode has been of sufficient severity and/or duration to cause significant changes in the metabolism and the structural integrity of heart muscle, reperfusion may paradoxically result in a worsening of heart function, out of proportion to the amount of dysfunction expected simply as a result of the duration of blocked flow. Although the beneficial effects of early reperfusion of ischemic myocardium with thrombolytic therapy, PTCA, or CABG are now well established, an increasing body of evidence indicates that reperfusion also induces an additional injury to ischemic heart muscle, such as the extension of myocardial necrosis, i.e., extended infarct size and impaired contractile function and metabolism. Hearts undergoing reperfusion after transplantation also undergo similar reperfusion injury events. In generally, all organs undergoing reperfusion are vulnerable to reperfusion injury.

Reperfusion injury can be defined as the damage that occurs to an organ that is caused by the resumption of blood flow after an episode of ischemia. This damage is distinct from the injury resulting from the ischemia per se. One hallmark of reperfusion injury is that it may be attenuated by interventions initiated before or during the reperfusion. Reperfusion injury results from several complex and interdependent mechanisms that involve the production of reactive oxygen species, endothelial cell dysfunction, microvascular injury, alterations in intracellular Ca²⁺ handling, changes in myocardial metabolism, and activation of neutrophils, platelets, cytokines and the complement system. All of the deleterious consequences associated with reperfusion constitute a spectrum of reperfusion-associated pathologies that are collectively called reperfusion injury. Reperfusion injury can extend not only acutely, but also over several days following the heart attack. Similar mechanisms of injury are observed in other organs and tissues that are subjected to ischemia and reperfusion.

Postconditioning is a method of treatment for significantly reducing reperfusion injury to an organ or tissue already undergoing total or subtotal ischemia, wherein the perfusion (blood flow) conditions are modified during the onset of reperfusion. Postconditioning is characterized by a series of brief, iterative interruptions in coronary-artery arterial reperfusion applied at the immediate onset of reperfusion. The bursts of reflow and subsequent occlusive interruptions last for a matter of seconds, ranging from 60 second intervals in larger animal models to 5-10 second intervals in smaller rodent models. Preliminary studies in humans used 1 minute intervals of reperfusion and subsequent interruptions in blood flow during catheter-based percutaneous coronary intervention (PCI).

Accordingly, what is needed but unavailable in the art, is a system and method of enhancing the beneficial effects of postconditioning to further reduce reperfusion injury in an organ or tissue undergoing total or subtotal ischemia without encountering the aforementioned disadvantages and challenges that exist in the conventional apparatuses and methods described above. In one aspect, provided herein is a system and method that can be performed as an adjunct to conventional reperfusion techniques, such as for example, PTCA reperfusion, which is increasingly a standard of care for patient's suffering an acute MI. One would also appreciate that the system and method described herein allows for general control of reperfusion and can be used in any desired modality in which control of reperfusion to a selected area of the subject.

### SUMMARY OF THE INVENTION

The goal of this invention is to overcome above-mentioned difficulties in designing and operating postconditioning systems in patients subsequent to an ischemic event.

In one embodiment, the present invention is directed to a system for preventing injury to an organ or tissue in a patient during or after reperfusion that comprises a means for modulating the flow of fluid within a vessel of the subject and a control system in operable control of the means for modulating the flow of fluid. In one aspect, the control system is selectively actuated to control the means for modulating the flow of fluid within the vessel of the subject to effect a pulsation of the flow of fluid within the vessel for a desired period of time and at a desired pulsatile frequency.

In a further embodiment, the system of the present invention comprises a means for delivering at least one drug. In one aspect, the control system is configured to control the means for delivering the at least one drug.

Still further, the system of the present invention can comprise a means for regulating the temperature of a fluid that is in communication with a catheter that is configured for selective insertion into a desired vessel of the patient. In this aspect, the control system is configured to be in operable communication with the means for regulating the temperature of the supplied fluid such that the temperature of the supplied fluid can be maintained at a desired temperature.

Related methods of operation are also provided. Other systems, methods, features, and advantages of the postconditioning system for use in reperfusion injury treatment modalities will be or will become apparent to one with skill in the art upon examination of the following figures and detailed description. It is intended that all such additional systems, methods, features, and advantages be included within this description, be within the scope of the postconditioning system for use in post-ischemic treatments, and be protected by the accompanying claims.

### BRIEF DESCRIPTION OF THE DRAWINGS

The accompanying drawings, which are incorporated in and constitute a part of this specification, illustrate certain aspects of the instant invention and together with the description, serve to explain, without limitation, the principles of the invention. Like reference characters used therein indicate like parts throughout the several drawings.

FIG. 1 is a schematic illustration of an exemplary flow rate control module of a postconditioning system of the present invention, according to one aspect of the present invention.

FIG. 2 is a schematic illustration of an exemplary balloon inflation module of a postconditioning system of the present invention, according to one aspect of the present invention.

FIG. 3 is a schematic illustration of an exemplary drug control module of a postconditioning system of the present invention, according to one aspect of the present invention.

FIG. 4 is a schematic illustration of an exemplary temperature control module of a postconditioning system of the present invention, according to one aspect of the present invention.

FIG. 5 is a schematic illustration of an exemplary balloon catheter positioned in the heart to effect postconditioning by modulating the inflation of the catheter.

FIG. 6 is a schematic illustration of an exemplary balloon catheter positioned in the heart to effect postconditioning by modulating flow of fluid exiting the distal end of the catheter.

FIG. 7 is a schematic illustration of an exemplary two balloon catheter positioned in the heart.

FIG. 8 is a cross-sectional view of an exemplary two occlusive balloon embodiment of a catheter having a central lumen that is configured to accommodate a desired flow or blood and/or other selected fluids for use in one exemplary embodiment of the present invention.

FIG. 9 shows the experimental protocol used to determine the effect of one possible variation in postconditioning on myocardium after ischemia (I) and reperfusion (R). Control group (n=10); Postconditioning (n=10); Pre-conditioning (n=9); Ischemic preconditioning was elicited by 5 minutes of coronary occlusion followed by 10 minutes of reperfusion before 60 minutes of left anterior descending coronary artery (LAD) occlusion, and postconditioning 3 cycles of 30 seconds of reperfusion followed by 30 seconds of occlusion before 3 hours of reperfusion, respectively.

FIG. 10 is a schematic illustration of one exemplary embodiment of a computing system/device that can be used to practice aspects of the present invention.

### DETAILED DESCRIPTION OF THE INVENTION

The present invention can be understood more readily by reference to the following detailed description, examples, drawings, and claims, and their previous and following description. However, before the present devices, systems, and/or methods are disclosed and described, it is to be understood that this invention is not limited to the specific devices, systems, and/or methods disclosed unless otherwise specified, as such can, of course, vary. It is also to be understood that the terminology used herein is for the purpose of describing particular aspects only and is not intended to be limiting.

The following description of the invention is provided as an enabling teaching of the invention in its best, currently known embodiment. To this end, those skilled in the relevant art will recognize and appreciate that many changes can be made to the various aspects of the invention described herein, while still obtaining the beneficial results of the present invention. It will also be apparent that some of the desired benefits of the present invention can be obtained by selecting some of the features of the present invention without utilizing other features. Accordingly, those who work in the art will recognize that many modifications and adaptations to the present invention are possible and can even be desirable in certain circumstances and are a part of the present invention. Thus, the following description is provided as illustrative of the principles of the present invention and not in limitation thereof.

As used herein, the singular forms “a,” “an” and “the” include plural referents unless the context clearly dictates otherwise. Thus, for example, reference to a “lumen” includes aspects having two or more such lumens unless the context clearly indicates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “optional” or “optionally” mean that the subsequently described event or circumstance may or may not occur, and that the description includes instances where said event or circumstance occurs and instances where it does not.

Referring now to the figures, the present invention provides, in one embodiment, a system for the reduction or prevention of injury to an organ or tissue of a subject before, during or after reperfusion. While the primary examples described herein are described in reference to the heart, it will be appreciated that the systems and methods of the present invention are not limited to application to a heart, but is intended to be used with any arterial or venous access to any tissue or organ that may be susceptible to reperfusion injury.

Reperfusion injury has been reported to extend the degree and severity of post-ischemic injury over about a twenty-four (24) to seventy-two (72) hour period after onset of reperfusion. The immediate reperfusion injury phase is initiated at the onset of reperfusion, and includes the initial burst of reactive oxygen species, activation of neutrophils, and vascular endothelial cells and their interaction, and opening of the mitochondrial permeability transition pore. This very early activation influences downstream pathyphysiological end points such as necrosis and apoptosis. Several signaling pathways may also be triggered during the early reperfusion period, including the reperfusion injury survival kinase (RISK) and pro-apoptotic pathways. Subacute events within several hours of reperfusion include release of inflammatory mediators, complement, etc. that may amplify the initial events and further exacerbate the injury. Late phase events may involve gene-based regulation of soluble injury mediators, which continue the pathogenesis of necrosis and apoptosis over the next about twenty-four (24) to seventy-two (72) hours.

The events occurring during the first minutes of reperfusion can predetermine such pathological end points as infarct size and apoptosis. It is the avoidance of these critical early events that is the foundation for the strategy of reperfusion therapeutics, as described herein with respect to various aspects of the present invention.

Aspects of the present invention provide systems and methods for controlling reperfusion by modifying the conditions and composition of the immediate reperfusion phase. In various aspects, modification of the conditions of reperfusion can involve mechanical maneuvers including but not limited to temperature control, pressure control, applying postconditioning, or a combination of one or all of these. The use of specific cell or cytokine filters to treat the reperfusate may also constitute a method to modify the initial reperfusate. In some aspects, modifying the composition of the reperfusate can comprise increasing osmolality of the reperfusate, making the reperfusate either alkolotic or acidotic, including adjunct drugs in single or combinational regimen, or a combination of these.

According to aspects of the present invention, a system is provided for preventing or attenuating injury to a cell, organ or tissue in a subject during or after reperfusion following an ischemic event to the cell, organ or tissue. Examples of organs that can be treated with postconditioning include, but are not limited to, the lung, liver, pancreas, heart and kidney. As used herein, a subject can include domesticated animals (such as, but not limited to, cats, dogs, etc.), livestock (such as, but not limited to, cattle, horses, pigs, sheep, goats, etc.), laboratory animals (such as, but not limited to, mice, rabbits, rats, guinea pigs, etc.), and birds. In some aspects, the subject is a mammal such as a primate, and, more preferably, is a human.

In various aspects, the system 20 of the present invention can comprise one or more of the following: an intralumenal flow rate control module 100, a balloon inflation module 200, a temperature control module 300, and drug control module 400. For example, in one aspect, the system 20 can comprise a means for modulating the flow of fluid within the vessel of the subject and a control system 22 configured to control the flow of the fluid within the vessel. In a further aspect, the control system 22 can be configured to control pulsation of the flow of fluid within the vessel of the subject for a desired period of time and at a desired pulsatile frequency.

Methods and means for controlling the pulsatile flow of fluid within a vessel for effecting a reduction in ischemic-reperfusion injury via postconditioning modalities are described in the applicant's co-pending U.S. patent application Ser. No. 10/499,052, which is herein fully incorporated by reference in its entirety. Further, methods of postconditioning reperfusion of an organ or tissue injured by ischemia in combination with the administration of one or more tissue protective agents that enhance the effect of postconditioning are described in applicant's International Publication No. WO 2006/069170, which is herein fully incorporated by reference in its entirety.

As will be appreciated by one skilled in the art, the preferred embodiment may be implemented as a method, a data processing system, or a computer program product. Accordingly, the preferred embodiment may take the form of an entirely hardware embodiment, an entirely software embodiment, or an embodiment combining software and hardware aspects. Furthermore, implementations of the preferred embodiment may take the form of a computer program product on a computer-readable storage medium having computer-readable program instructions (e.g., computer software) embodied in the storage medium. More particularly, implementations of the preferred embodiments may take the form of web-implemented computer software. Any suitable computer-readable storage medium may be utilized including hard disks, CD-ROMs, optical storage devices, or magnetic storage devices.

The preferred embodiments according to the present invention are described below with reference to block diagrams and flowchart illustrations of methods, apparatuses (i.e., systems) and computer program products according to an embodiment of the invention. It will be understood that each block of the block diagrams and flowchart illustrations, and combinations of blocks in the block diagrams and flowchart illustrations, respectively, can be implemented by computer program instructions. These computer program instructions may be loaded onto a general purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks. Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and computer instructions.

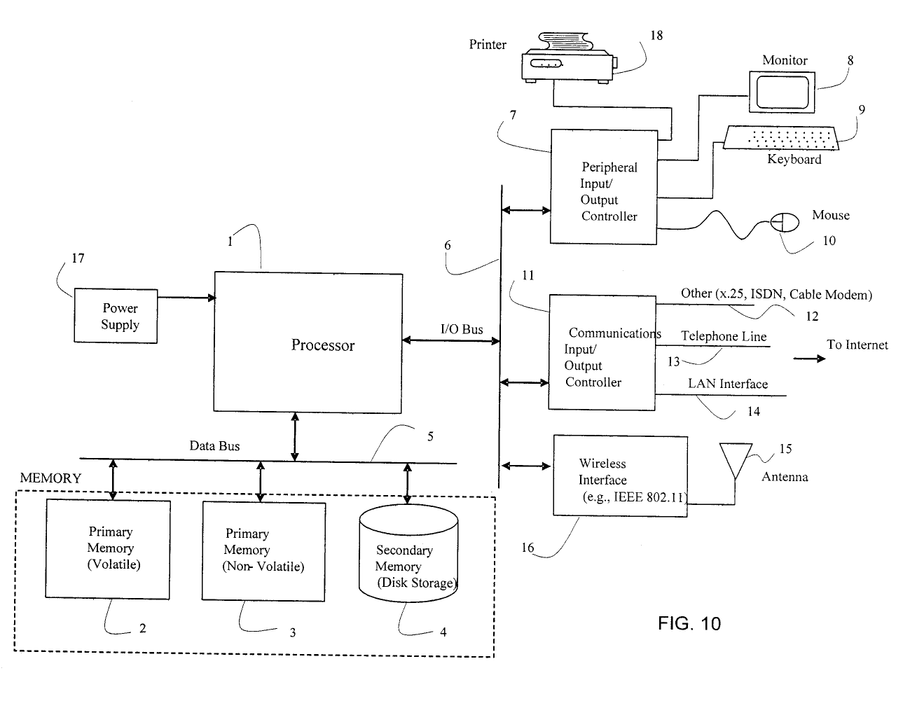

In the preferred embodiments referenced herein, a “computer” or “computing device” may be referenced. Such computer may be, for example, a mainframe, desktop, notebook or laptop, a hand held device such as a data acquisition and storage device, or it may be a processing device embodied within another apparatus. In some instances the computer may be a “dumb” terminal used to access data or processors over a network. Turning to FIG. 10, one embodiment of a computing device is illustrated that can be used to practice aspects of the preferred embodiment. In FIG. 10, a processor 1, such as a microprocessor, is used to execute software instructions for carrying out the defined steps. The processor receives power from a power supply 17 that also provides power to the other components as necessary. The processor 1 communicates using a data bus 5 that is typically 16 or 32 bits wide (e.g., in parallel). The data bus 5 is used to convey data and program instructions, typically, between the processor and memory. In the present embodiment, memory can be considered primary memory 2 that is RAM or other forms which retain the contents only during operation, or it may be non-volatile 3, such as ROM, EPROM, EEPROM, FLASH, or other types of memory that retain the memory contents at all times. The memory could also be secondary memory 4, such as disk storage, that stores large amount of data. In some embodiments, the disk storage may communicate with the processor using an I/O bus 6 instead or a dedicated bus (not shown). The secondary memory may be a floppy disk, hard disk, compact disk, DVD, or any other type of mass storage type known to those skilled in the computer arts.

The processor 1 also communicates with various peripherals or external devices using an I/O bus 6. In the present embodiment, a peripheral I/O controller 7 is used to provide standard interfaces, such as RS-232, RS422, DIN, USB, or other interfaces as appropriate to interface various input/output devices. Typical input/output devices include local printers 18, a monitor 8, a keyboard 9, and a mouse 10 or other typical pointing devices (e.g., rollerball, trackpad, joystick, etc.).

The processor 1 typically also communicates using a communications I/O controller 11 with external communication networks, and may use a variety of interfaces such as data communication oriented protocols 12 such as X.25, ISDN, DSL, cable modems, etc. The communications controller 11 may also incorporate a modem (not shown) for interfacing and communicating with a standard telephone line 13. Finally, the communications I/O controller may incorporate an Ethernet interface 14 for communicating over a LAN. Any of these interfaces may be used to access a wide area network such as the Internet, intranets, LANs, or other data communication facilities.

Finally, the processor 1 may communicate with a wireless interface 16 that is operatively connected to an antenna 15 for communicating wirelessly with another device, using for example, one of the IEEE 802.11 protocols, 802.15.4 protocol, or a standard 3G wireless telecommunications protocols, such as CDMA2000 1×EV-DO, GPRS, W-CDMA, or other protocol.

In one aspect, the system 20 can comprise an access catheter 30 that is configured for insertion in a vessel of a subject, such as, for example and not meant be limiting, angioplasty catheters with or without stents, peripheral balloon catheters that can accommodate flow through a central or other lumen, and the like. In one exemplary aspect, the catheter 30 can comprise a first lumen 32 in communication with a distal end 34 of the catheter and a distal region comprising at least one expandable member 36 (such as, but not limited to, a balloon) that is movable from and between a non-inflated position, in which fluid can pass by the at least one expandable member, and a blocking position, in which the at least one expandable member is inflated to occlude the vessel such that fluid is prevented from passing by the at least one expandable member.

In a further aspect, the intralumenal flow rate control module 100 can be implemented in the control system 22 and can be is configured to control the flow of fluid within the vessel. In various aspects, the flow rate control module can be used to control attributes of intravascular perfusion with fluids, blood, or blood/fluid combinations, with or without adjunct drugs. As one skilled in the art will appreciate, control of flow rate is important for various reasons, such as, for example, supplying adequate perfusion, preventing over-perfusion, and, in some aspects, to adequately deliver drugs.

It is contemplated that the control system 22 can be manually controlled or automatically controlled, or be controlled by a combination of manual and automatic control. Optionally, the control system 22 may comprise one or more control algorithms that are configured to automatically control the means for modulating the flow of fluid. The algorithm may include implementing one or more flow patterns, such as, for example and not meant to be limiting, constant flow, gradual reperfusion, waveform flow (for example, sine wave, square wave, sawtooth wave, and the like), or other flow patterns. The amplitude, duration, or other aspect of these flow patterns can also be controlled via the algorithm. In constant flow, flow rate is held constant without pulsation at a target or manually chosen pressure or flow rate. In a further aspect, the flow rate can also be adjusted to achieve a predetermined intravascular pressure that can be sensed by a pressure sensor positioned proximate the distal or outlet end of the catheter.

In an exemplary aspect, the intralumenal flow rate can be manually or automatically set in the range of from about 0 mL/minute to about 100 mL/minute, to include the flow rates of 5, 10, 15, 20, 25, 30, 35, 40, 45, 50, 55, 60, 65, 70, 75, 80, 85, 90, and 95 mL/minute. Optionally, flow rate can be set in the range of from about 0 mL/minute to about 200 mL/minute, to include the flow rates of 5, 10, 15, 20, 25, 30, 35, 40, 45, 50, 55, 60, 65, 70, 75, 80, 85, 90, 95, 105, 110, 115, 120, 125, 130, 135, 140, 145, 150, 155, 160, 165, 170, 175, 180, 185, 190, and 195 mL/minute.

In a gradual reperfusion embodiment, a time interval over which the reperfusion is gradually increased can be set. In this aspect, the trajectory of the intralumenal flow rate increase can be linear or exponential. In another aspect, the gradual reperfusion can be implemented until a target pressure or flow rate is achieved. In one aspect, pressure sensed by the pressure sensor mounted proximate the distal end of the catheter can be compared to the predetermined target pressure. In an optional waveform flow embodiment, various waveforms can be chosen, such as a sine waveform, fully pulsatile waveform (e.g., a square wave) which varies between full flow and no- or low-flow options, or a sawtooth type flow on-off waveform.

As described above, intralumenal flow can be set or adjusted based on the predetermined target pressure. In various aspects, pressure can be either peripheral pressure or distal pressure measured within the vessel from the catheter 30, which in some aspects is equipped with one or more conventional pressure sensors. In one aspect, the system comprises a pressure sensor 42 mounted to a distal end portion of the catheter 30. In another aspect, the system comprises a pressure sensor 44 positioned within a vessel of the subject remote from the catheter. In other aspects, the control system 22 comprises both of these pressure sensors. The one or more pressure sensors are in operative communication with the control system 22.

In one contemplated aspect, if at least two pressure sensors (such as those described above) are used to exemplarily sample a distal intravascular pressure and a proximal femoral arterial pressure, the control system 22 can compare the pressures sensed at the respective sensors to a target pressure to derive a comparative pressure result and control the intralumenal flow of fluid exiting the first lumen of the catheter in response to the comparative pressure result. The sensed pressure differential between the respective distal and proximal pressure sensors defines the flow rate and/or pressure that well be generated over a given period of time and/or over a given waveform. Optionally, if only one pressure sensor is used, the control system 22 can compare the pressure sensed at the single pressure sensor to a target pressure to derive a comparative pressure result and to control the flow of the fluid exiting the first lumen of the catheter in response to the comparative pressure result.

In a further aspect, the means for modulating the intralumenal flow of fluid may comprise a source of pressurized fluid in communication with the first lumen of the catheter. The control system 22 can thus be configured to modulate the intralumenal flow of the fluid exiting the first lumen of the catheter to control the pulsatile flow of fluid within the vessel.

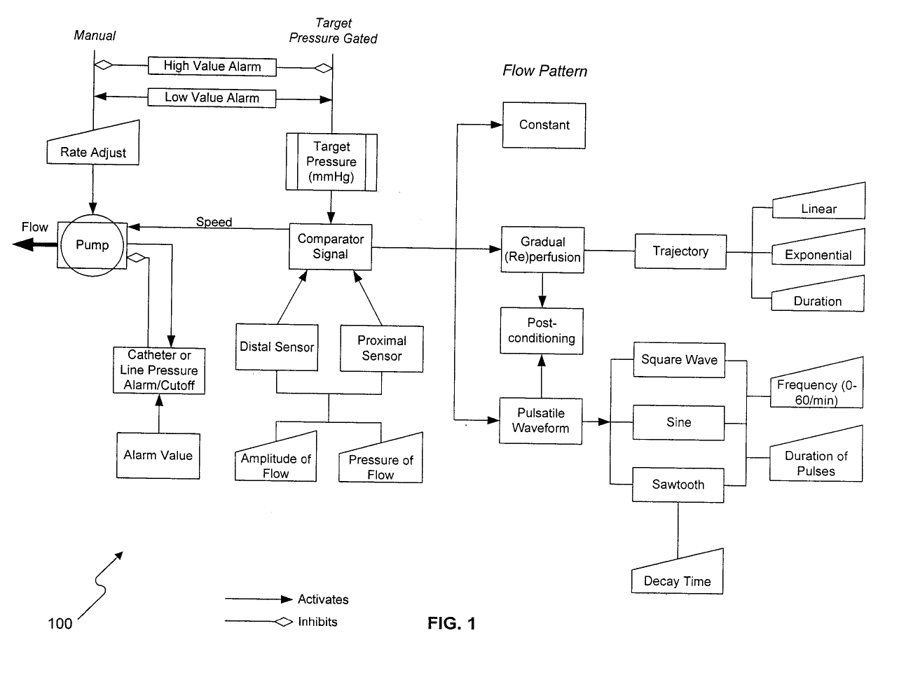

FIG. 1 illustrates various aspects of the intralumenal flow control module 100. As can be seen, and as described in part above, the intralumenal flow pattern can be one or a combination of constant flow, gradual reperfusion, or pulsatile waveform, among other patterns. If gradual reperfusion is chosen, the trajectory (such as, but not limited to, linear or exponential) of the flow can be chosen, as well as the duration of the trajectory. The duration of the trajectory may be selected in seconds, minutes, hours, or other measure of time. In one aspect, the duration can be in the range of about one (1) minute to about one hundred twenty (120), or, optionally, in the range of about two (2) to about sixty (60) minutes. Optionally, if a pulsatile waveform is chosen, a pattern of square wave, sawtooth wave, sine wave, or a combination of one or more can be selected. The frequency of the pulsatile waveform (such as, for example and not meant to be limiting, 0-60 per minute) or duration of the pulses can be selected, as well as the attack and the decay time for the sawtooth waveform.

As shown in FIG. 1, in one aspect the amplitude of the intralumenal flow can range from about 0 to about 120 mL/minute, or a manual or target pressure can be set for the flow rate. One or more conventional pressure sensors (such as a distal and proximal pressure sensor) can be configured to measure pressure of the flow. A comparator signal is used to compare these pressures to a target pressure (such as a pressure selected from the range of about 0 mmHg to about 120 mmHg).

In one aspect, an alarm can be activated if the pressures sensed by the distal and proximal sensors are lower in value or higher in value than the target pressure. In this aspect, the control system 22 can be configured to automatically respond to the alarm and adjust the flow rate and/or pattern. In other aspects, the flow rate or pattern can be adjusted manually. Optionally, the control system 22 is configured to respond to the alarm by cutting off flow or allowing full flow, depending on the alarm that was activated. As one skilled in the art will appreciate, a conventional pump can be configured to provide flow in response to the settings described above. It is also contemplated that the auto-control function of the control system 22 can be configured to operate independent of any alarm feature.

In one aspect, and as briefly described above, the means for modulating the flow of fluid can comprise at least one expandable member 36, such as a balloon positioned at the distal end portion of a catheter 30. The balloon inflation module 200 of the present invention can be configured to control the inflation and deflation of the balloon, via the control system 22, to thereby control the pulsatile flow of fluid within the vessel past the balloon of the catheter. The actual design of the expandable member inflation/deflation system can be implemented using known hydraulic and pneumatic elements. Further, the cyclical process of inflation/deflation of the expandable member can be automated and implemented in the control system 22 using known technology.

In various aspects, parameters of the balloon inflation module can be controlled manually, automatically, or a combination of both. In one aspect, the inflation and deflation of the expandable member can be activated in an algorithm, which can be programmable, that specifies duration, number of cycles, and other attributes. Optionally, a user can choose or set the duration of each inflation, the duration of each deflation, and the number of cycles as digital or analog settings, or from a menu of preset options. In this aspect, it is contemplated that the user can manually program a desired algorithm. It is further contemplated that the control system can be configured to control the frequency of the cycle of movement of the expandable member between the non-inflated position and blocking position to obtain a desired pulsatile frequency of flow through the vessel.

In one exemplary aspect, the frequency of the cycle of movement of the expandable member can comprise successive periods of n seconds (or other unit of time measurement) of inflation and m seconds (or other unit of time measurement) of deflation. In one aspect, the control system 22 can be configured to control the pressure of the expandable member (such as a balloon) during the inflation mode. In some aspects, the control system can control the pressure such that a target pressure is achieved in the expandable member. As one will appreciate, this embodiment of the system 20 contemplates that the balloon can be selectively deflated to allow for reperfusion of the selected area and then is inflated to occlude the vessel—this postconditioning cycle is repeated as desired to affect the desired pulsatile flow of fluid within the vessel.

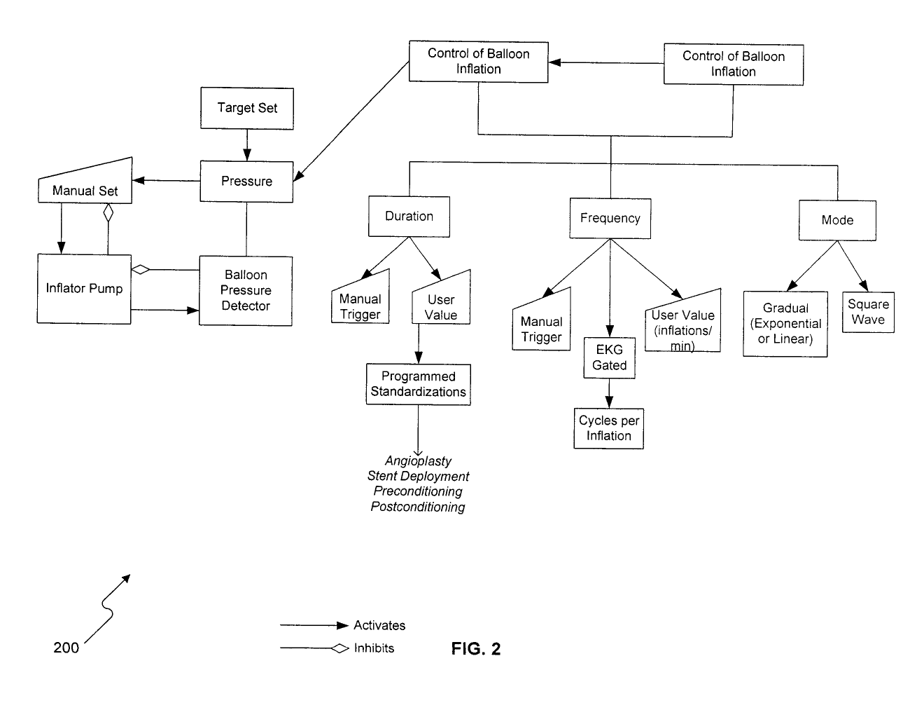

It is further contemplated that the expandable member can be, in one example, expanded at a higher pressure initially to effect a disruption of the occlusion and to open the vessel. Optionally, in subsequent postconditioning cycles, the expandable member can be inflated to a lower inflation pressure than that reached in the initial opening of the occlusion to reduce potential injury to the artery from over dilation of the vessel. In this aspect, the selected lower inflation pressure is sufficient to achieve sufficient occlusion of the vessel to cause effective termination of blood flow to the distal branches of the vessel. FIG. 2 illustrates aspects of an exemplary balloon inflation module 200. As can be seen in FIG. 2, and as described in part above, the balloon inflation module can be utilized to control inflation and deflation of one or more balloons or occluding devices. The duration of inflation can be set manually, and thereafter implemented automatically based on the setting. The duration of inflation can be set in various increments of time, such as seconds, minutes, or hours. For example, the duration of inflation can be selected from the range of about 0 seconds to about 600 seconds, optionally from the range of about 0 seconds to about 360 seconds, to include the durations of 10, 20, 30, 40, 50, 60, 70, 80, 90, 100, 11, 120, 130, 140, 150, 160, 170, 180, 190, 200, 210, 220, 230, 240, 250, 260, 270, 280, 290, 300, 310, 320, 330, and 340 seconds. Various duration settings can be programmed, so as to standardize inflation time during deployment of stents, performance of angioplasties, preconditioning and postconditioning.

In one aspect, balloon inflation can also be fully controlled manually. Optionally, if an algorithm-controlled inflation is selected, the frequency of inflation and deflation can also be set. In one exemplary aspect, the frequency can be selected from the range of about 0.5 inflation per minute to about 80 inflations per minute. It is also contemplated that the inflation and deflation times can be chosen in seconds. In one aspect, the frequency of inflation and deflation cycles can also be fully controlled manually.

In a further aspect, the mode of inflation/deflation can also be selected, including gradual inflation or deflation (which can be exponential or linear), square wave (rapid transition between inflation and deflation), or another algorithm. As described above, pressure of the expandable member while in the inflated (or blocking) position can be set (e.g., as a target pressure), or can be monitored manually. In one aspect, the target pressure can be set in a range between about 0 atm to about 12 atm of pressure. An additional pressure can be used to monitor pressure of the expandable member. A conventional inflator pump [not shown] can be configured to respond to the automatic settings and/or manual changes and respond by inflating or deflating the expandable member accordingly.

As described above, in one aspect, the means for modulating the flow of fluid can comprise a source of pressurized fluid 40 in communication with the lumen of the catheter 30. In this aspect, the control system 22 can be configured to modulate the intralumenal flow of fluid exiting the first lumen of the catheter into the selected vessel of the subject to control the pulsatile flow of fluid within the vessel during time intervals when the expandable member is in the blocking position.

Optionally, the control system 22 can be configured to control more than one balloon (such as two balloons) positioned along a catheter. In one exemplary aspect, a second balloon can be used to create an area for dwelling of solutions, such as angiogenic factors, drug-impregnated liposomes, drug impregnated nanoparticles, viral vectors or viral-associated vectors, or any drug or agent that requires a period of stasis for absorption in a chamber that can be defined within the vessel between the two inflated expandable members. In another aspect, the created area of dwelling can also be used to isolate and perfuse an arterial or venous branching off the catheterized vessel, e.g., perforator arteries off LAD or the renal artery, the renal vein off the vena cava. Such a catheter and balloon configuration is described in Applicant's co-pending U.S. patent application Ser. No. 10/493,779, which is herein fully incorporated by reference in its entirety. The exemplified catheter can supply blood (or other fluid) flow through the first lumen, as described above, during the dwell time when one or both expandable members are inflated, so as to prevent additional ischemia of the distal organ or tissue.

In some aspects, the system 20 can also comprise a means for delivering at least one drug to the subject. In one aspect, the control system 22 can be configured to control the delivery of the at least one drug. In one aspect, the control system controls the delivery of the drug(s) via the drug control module 300. In one aspect, a plurality of drugs can be delivered to the subject and the control system controls the delivery of each respective drug. It is contemplated that the plurality of drugs can be delivered substantially simultaneously or substantially sequentially. Optionally, some of the plurality of drugs can be delivered simultaneously and some of the drugs can be delivered sequentially. The one or more drugs can be delivered via the catheter (such as via the first lumen, or via a separate drug-delivery lumen) during the reperfusion process.

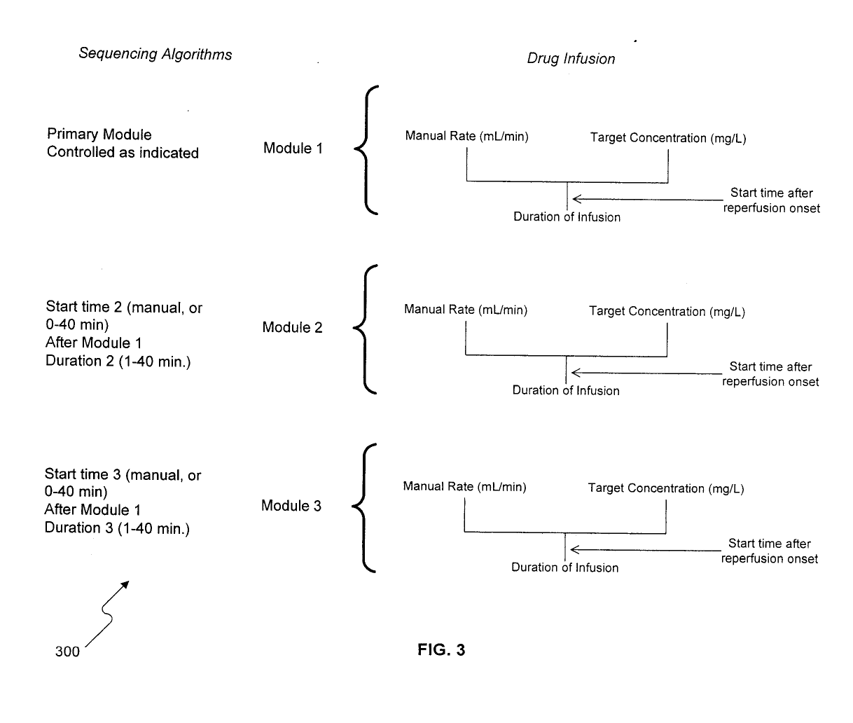

FIG. 3 illustrates aspects of an exemplary drug control module 300. As can be seen, various aspects of the drug delivery sequence can be controlled, via the control system 22, either manually, automatically, or a combination of both. For example, the start time, infusion rate, and/or target concentration of each drug can be controlled. As described above, drugs can be delivered simultaneously (i.e., adenosine, sodium-hydrogen exchange inhibitor, and the like) and/or sequentially (i.e., adenosine for X minutes, followed by L-arginine for Y minutes). In another aspect, drugs can be delivered at the same time that other maneuvers (such as inflation/deflation of an expandable member, and/or adjustment of the intralumenal flow rate) are applied. For example, postconditioning may be applied by either inflating the expandable member (such as a balloon) or delivering pulsatile perfusion, while simultaneously delivering a drug that enhances the postconditioning effect (i.e., adenosine, cariporide, L-arginine, etc.). FIG. 3 illustrates three exemplary module sequences of the drug control module. In each module, a rate of drug infusion can be manually selected from the exemplary range of about 0 mL/minute to about 120 mL/minute. Optionally, the target concentration (in mg/L) can be set. In another aspect, the duration of infusion as well as the start time after the onset of reperfusion can be selected.

In various aspects, the system further comprises a means for regulating the temperature of a pressurized supplied fluid in communication with the first lumen 32 of the catheter 22. In this aspect, the control system 22 can be configured to control the means for regulating the temperature such that the temperature of the fluid being supplied to the first lumen is at a desired temperature.

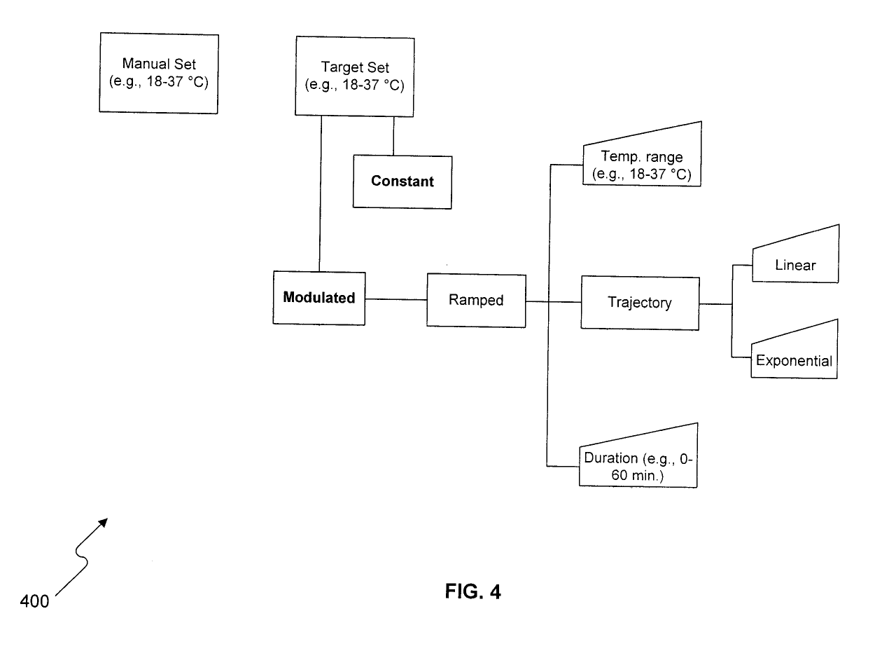

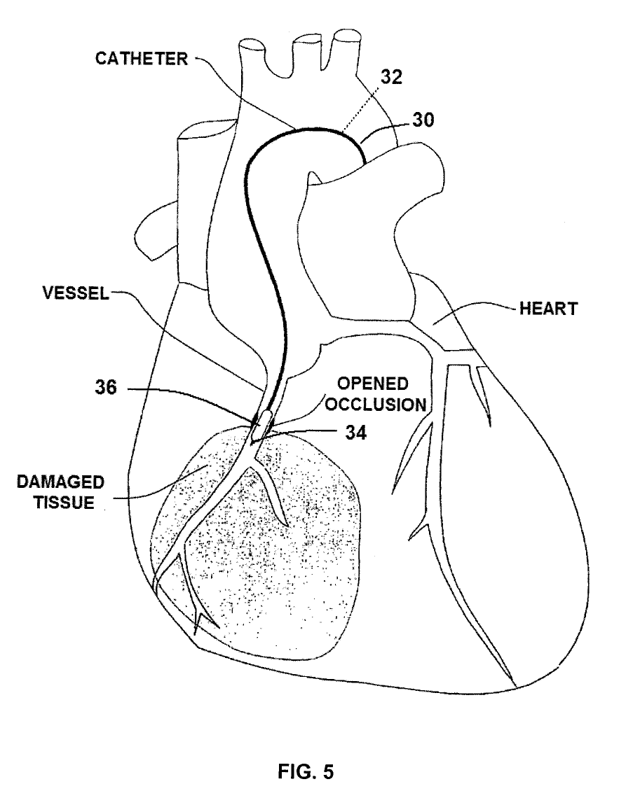

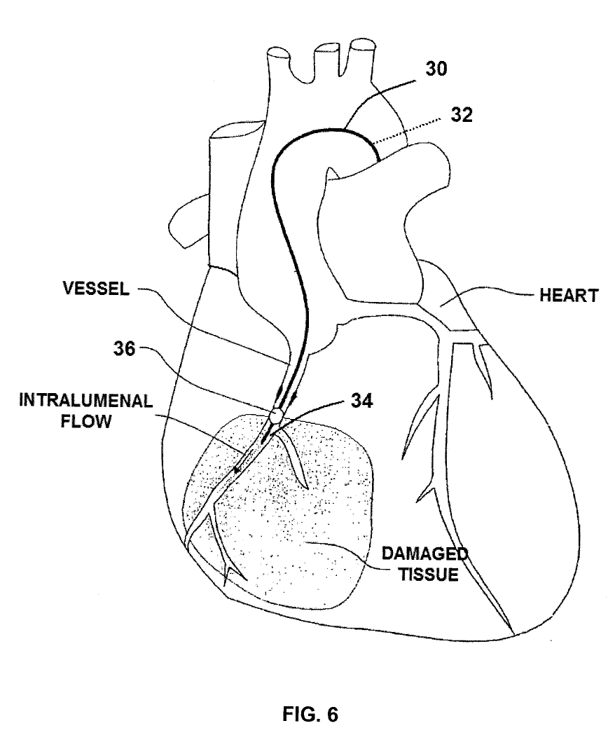

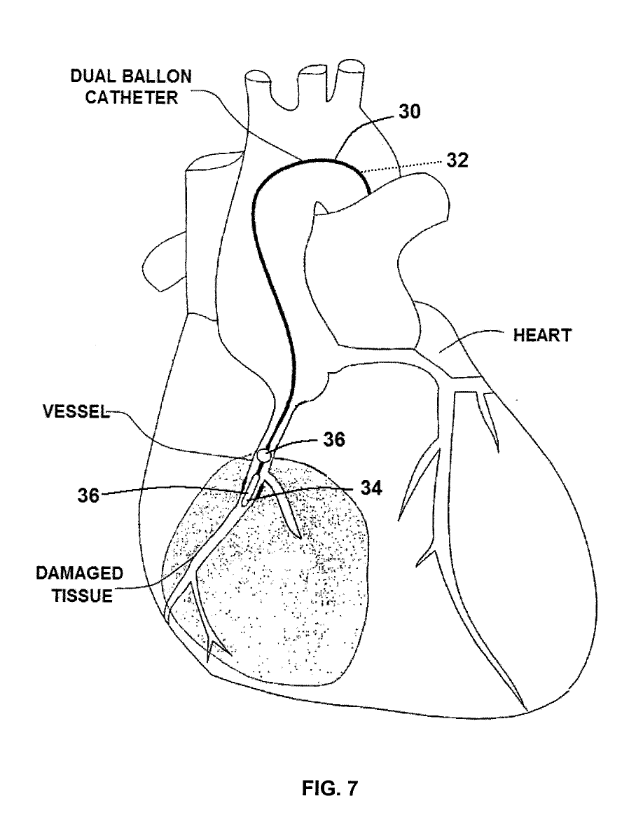

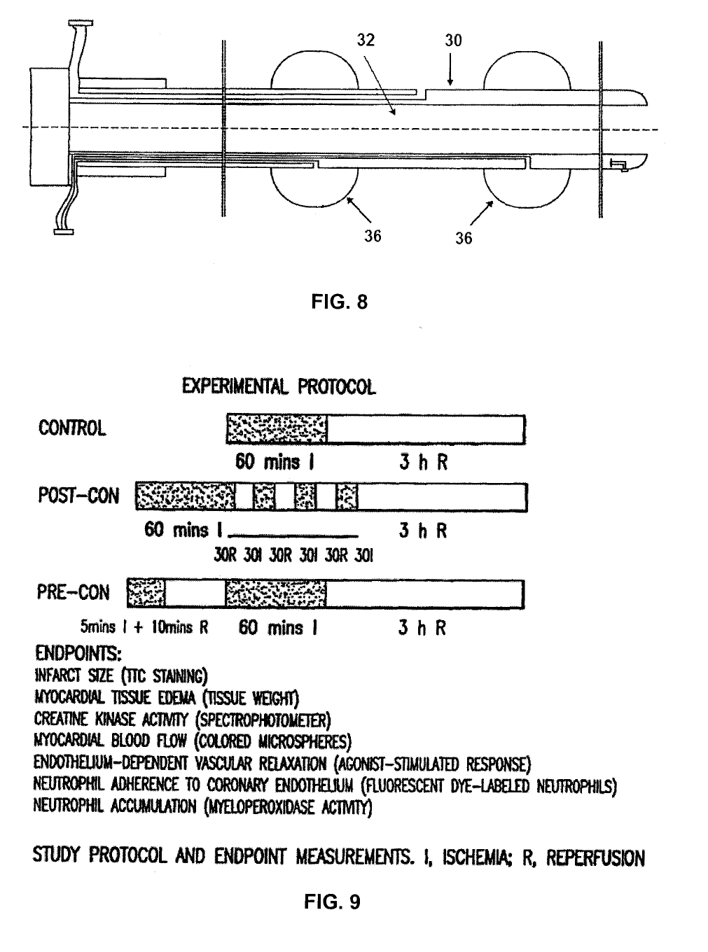

In one aspect, the supplied fluid at the distal end of the first lumen can be held substantially constant between about 18° C. and about 37° C. Optionally, the control system can be configured to control the means for regulating the temperature to decrease the temperature of the supplied fluid at the distal end of the first lumen toward a target temperature range of between about 18° C. to about 37° C. over a predetermined period of time. In an additional aspect, the control system 22 can control the means for regulating the temperature of the supplied fluid to increase the temperature of the supplied fluid from the target temperature range over time. As can be seen in FIG. 4, which illustrates an exemplary temperature control module 400, temperature can be manually set and manually controlled, or a target temperature (or temperature range) can be set and automatically monitored and controlled. If the temperature is to be modulated, the rate of decrease and/or increase in perfusate temperature can be controlled by algorithms. The modulated temperature can be ramped either up or down, and the ramping characteristics can be linear or exponential. The duration of the ramping trajectory can also be programmed. As may be appreciated, a typical heat exchanger as known in the art can be used to control the temperature of the fluid flow to the first lumen of the catheter.

Aspects of the present invention also provide a method for preventing injury to an organ or tissue in a subject during or after reperfusion following an ischemic event to the organ or tissue. In one aspect, the method comprises providing a means for modulating the flow of fluid within a vessel of the subject, providing a control system in operable control of the means for modulating the flow of fluid, and actuating the control system to control the means for modulating the flow of fluid within the vessel of the subject to effect a pulsation of the flow of fluid within the vessel for a desired period of time and at a desired pulsatile frequency. In one aspect, at least a portion of the control system can be controlled manually. Optionally, a portion of the control system can also be controlled automatically. For example, the control system in one aspect comprises at least one control algorithm adapted to control the means for modulating the flow of fluid.

In one aspect, a catheter is provided that is configured for insertion in the vessel of the subject. As described above, the catheter can comprise a first lumen in communication with a distal end of the catheter and a distal region comprising at least one expandable member. The at least one expandable member is movable from and between a non-inflated position, in which fluid can pass the at least one expandable member, and a blocking position, in which the expandable member is inflated to occlude the vessel such that fluid is prevented from passing by the at least one expandable member. In one aspect, the means for modulating the flow of fluid comprises the at least one expandable member. The control system can thus modulate the expandable member to control the pulsatile flow of fluid, for example blood, that flows past the expandable member of the catheter.

The control system can also control the pressure of the expandable member such that a target pressure is achieved in the expandable member. As described above, the control system can also control the frequency of the cycle of movement of the expandable member between the non-inflated position and the blocking position to achieve the desired pulsatile frequency. The frequency of the inflation/deflation cycle can include periods of inflation and periods of deflation, such as successive periods of n seconds of inflation and m seconds of deflation. Various modes of inflation and deflation can also be manually set and thereafter controlled by the control system.

In one aspect, the means for modulating the flow of fluid comprises a source of pressurized fluid in communication with the first lumen of the catheter. In this aspect, the control system is configured to modulate the intralumenal flow of fluid exiting the first lumen of the catheter during time intervals when at least one expandable member is in the blocking position. The flow of fluid through the first lumen can be controlled by the control system to maintain the flow of fluid at substantially a target pressure. In one aspect, the vessel blood flow is blocked by one expandable member of the catheter and the flow of fluids through the central lumen of the artery and hence into the vessel of the subject is controlled by the system. As described above, the control system 22 of the present invention can, in various aspects, control flow rate, pulsative characteristics, pulsative duration and frequency, temperature, and the additional of selected drugs.

As described above, the flow of fluid through the first lumen can have various rates, including constant flow, increasing flow, or decreasing flow, and can be in various waveforms, such as sinusoidal, sawtooth, or square. Thus, the flow rate of fluid can be increased to achieve a target pressure. Optionally, the flow rate can be substantially constant (such as for the purpose of maintaining a target pressure). It is contemplated that a target flow rate can be set and the control rate can increase or decrease the intralumenal flow rate of the fluid over time until the target flow rate is reached.

In one aspect, the method further comprises providing a means for delivering at least one drug. In this aspect, the control system is configured to control the means for delivering at least one drug. In some aspects, a plurality of drugs can be delivered to the subject, and the control system may be configured to control the delivery of each respective drug of the plurality of drugs. The control system, in some aspects, is configured to deliver the plurality of drugs substantially simultaneously. Optionally, the control system can be configured to deliver the plurality of drugs substantially sequentially. In another aspect, the control system can be configured to deliver at least some of the plurality of drugs substantially simultaneously and at least some of the plurality of drugs substantially sequentially.

In some aspects, the method of the present invention comprises providing a pressurized supplied fluid in communication with the first lumen of the catheter and providing a means for regulating the temperature of the supplied fluid. In this aspect, the control system is in operable communication with the means for regulating the temperature of the supplied fluid. Thus, in some aspects, the control system is activated to modulate the temperature of the supplied fluid. The control system can control the means for regulating the temperature to maintain a specific temperature, or to alter temperature, such as in a specific temperature range. For example, the control system can maintain the temperature of the supplied fluid at the distal end of the first lumen substantially in the range of about 18° C. to about 37° C. Optionally, the control system can control the means for regulating the temperature to decrease the temperature of the supplied fluid at the distal end of the first lumen toward a target temperature or temperature range, such as 18° C. to about 37° C., over a period of time. In other aspects, the control system controls the means for regulating the temperature to increase the temperature of the supplied fluid from the target temperature range.

It will be apparent to those skilled in the art that various modifications and variations can be made in the present invention without departing from the scope or spirit of the invention. Other aspects of the invention will be apparent to those skilled in the art from consideration of the specification and practice of the invention disclosed herein. It is intended that the specification and examples be considered as exemplary only, with a true scope and spirit of the invention being indicated by the following claims.

### REFERENCES

(1) Reimer K A, Jennings R B. The “wavefront phenomenon” of myocardial ischemic cell death. II. Transmural progression of necrosis within the framework of ischemic bed size (myocardial at risk) and collateral flow. Lab Invest 1979; 40(6):633-644.
(2) Reimer K A, Lowe J E, Rasmussen M M, Jennings R B. The wavefront phenomenon of ischemic cell death. 1. Myocardial infarct size vs duration of coronary occlusion in dogs. Circulation 1977; 56(5):786-794.
(3) Piper H M, Garcia-Dorado D, Ovize M. A fresh look at reperfusion injury. Cardiovasc Res 1998; 38:291-300.
(4) Piper H M, Meuter K, Schafer C. Cellular mechanisms of ischemia-reperfusion injury. Ann Thorac Surg 2003; 75:S644-S648.
(5) Jordan J E, Zhao Z-Q, Vinten-Johansen J. The role of neutrophils in myocardial ischemia-reperfusion injury. Cardiovas Res 1999; 43:860-878.
(6) Vinten-Johansen J. Reperfusion injury: Idle curiosity or therapeutic vector? J. Thrombosis Thrombolysis 1997; 4:59-61.
(7) Vinten-Johansen J. Involvement of neutrophils in the pathogenesis of lethal myocardial reperfusion injury. Cardiovasc Res 2004; 61:481-497.
(8) Zhao Z-Q, Nakamura M, Wang N-P, Velez D A, Hewan-Lowe K O, Guyton R A et al. Dynamic progression of contractile and endothelial dysfunction and infarct extension in the late phase of reperfusion. J Surg Res 2000; 94:1-12.
(9) Zhao Z-Q, Velez D A, Wang N-P, Hewan-Lowe K O, Nakamura M, Guyton R A et al. Progressively developed myocardial apoptotic cell death during late phase of reperfusion. Apoptosis 2001; 6(4):279-290.
(10) Piper H M, Schafer A C. The first minutes of reperfusion: a window of opportunity for cardioprotection. Cardiovasc Res 2004; 61:365-371.
(11) Piper H M, Garcia-Dorado D. Prime cause of rapid cardiomyocyte death during reperfusion. Ann Thorac Surg 1999; 68:1913-1919.
(12) Zhao Z-Q, Nakamura M, Wang N-P, Wilcox J N, Katzmark S, Guyton R A et al. Infarct extension and dynamic coronary endothelial dysfunction in the late reperfusion phase. Circulation 98[17], 1-796. 1998.
(13) Hausenloy D J, Mocanu M M, Yellon D M. Ischemic Preconditioning Protects by Activating Pro-Survival Kinases at Reperfusion. Am J Physiol Heart Circ Physiol 2005; 288:H971-H976.
(14) Hausenloy D J, Yellon D M. New directions for protecting the heart against ischaemia-reperfusion injury: targeting the Reperfusion Injury Salvage Kinase (RISK)-pathway. Cardiovasc Res 2004; 61(3):448-460.
(15) Lefer A M, Ma X-L, Weyrich A, Lefer D J. Endothelial dysfunction and neutrophil adherence as critical events in the development of reperfusion injury. Agents Actions (Suppl) 1993; 41:127-135.
(16) Kin H, Zhao Z-Q, Sun H-Y, Wang N-P, Corvera J S, Halkos M E et al. Postconditioning attenuates myocardial ischemia-reperfusion injury by inhibiting events in the early minutes of reperfusion. Cardiovasc Res 2004; 62:74-85.
(17) Sato H, Jordan J E, Zhao Z-Q, Sarvotham S S, Vinten-Johansen J. Gradual reperfusion reduces infarct size and endothelial injury but augments neutrophil accumulation. Ann Thorac Surg 1997; 64:1099-1107.
(18) Vinten-Johansen J, Lefer D J, Nakanishi K, Johnston W E, Brian C A, Cordell A R. Controlled coronary hydrodynamics at the time of reperfusion reduces postischemic injury. Cor Art Dis 1992; 3:1081-1093.
(19) Gunnes S, Ytrehus K, Sorlie D, Helgesen K G, Mjos O D. Improved energy preservation following gentle reperfusion after hypothermic, ischemic cardioplegia in infarcted rat hearts. Eur J Cardiothorac Surg 1987; 1:139-143.
(20) Okamoto F, Allen B S, Buckberg G D, Bugyi H, Leaf J. Studies of controlled reperfusion after ischemia. XIV. Reperfusion conditons: Importance of ensuring gentle versus sudden reperfusion during relief of coronary occlusion. J Thorac Cardiovasc Surg 1986; 92:613-620.
(21) Halkos M E, Kerendi F, Corvera J S, Wang N-P, Kin H, Payne C S et al. Myocardial protection with postconditioning is not enhanced by ischemic preconditioning. Ann Thorac Surg 2004; 78(3):961-969.
(22) Kin H, Zatta A J, Lofye M T, Amerson B S, Halkos M E, Kerendi F et al. Postconditioning reduces infarct size via adenosine receptor activation by endogenous adenosine. Cardiovasc Res. In press.
(23) Vinten-Johansen J, Zhao Z-Q, Zatta A J, Kin H, Halkos M E, Kerendi F. Postconditioning a new link in nature's armor against myocardial ischemia reperfusion injury. Bas Res Cardiol 2005; 100:1-16.
(24) Zhao Z-Q, Corvera J S, Wang N-P, Guyton R A, Vinten-Johansen J. Reduction in infarct size and preservation of endothelial function by ischemic postconditioning: Comparison with ischemic preconditioning. Circulation (Suppl) 106[19], 11314. 2002.

What is claimed is:

1. A method for preventing injury to an organ or tissue in a subject during or after reperfusion following an ischemic event to the organ or tissue, comprising:
providing a means for modulating the flow of fluid within a vessel of the subject;
providing a control system in operable control of the means for modulating the flow of fluid; and
actuating the control system to control the means for modulating the flow of fluid within the vessel of the subject to effect a pulsation of the flow of fluid within the vessel for a desired period of time and at a desired pulsatile frequency.

2. The method of claim 1, wherein at least a portion of the control system is controlled manually.

3. The method of claim 1, wherein the control system comprises at least one control algorithm that is adapted to control the means for modulating the flow of fluid.

4. The method of claim 1, further comprising providing a catheter configured for insertion therein the vessel of the subject, wherein the catheter comprises a first lumen in communication with a distal end of the catheter and a distal region comprising at least one expandable member that is movable from and between a non-inflated position, in which fluid can pass the at least one expandable member, and a blocking position, in which the at least one expandable member is inflated to occlude the vessel such that fluid is prevented from passing by the at least one expandable member.

5. The method of claim 4, wherein the means for modulating the flow of fluid comprises the at least one expandable member, wherein the control system is configured to modulate the at least one expandable member to control the pulsatile flow of fluid past the at least one expandable member of the catheter.

6. The method of claim 5, wherein the control system controls the pressure of the at least one expandable member such that a target pressure is achieved therein the at least one expandable member.

7. The method of claim 5, wherein the control system controls the frequency of the cycle of movement of the at least one expandable member between the non-inflated position and the blocking position to achieve the desired pulsatile frequency.

8. The method of claim 7, wherein the frequency of the cycle of movement is successive periods of n seconds of inflation and m seconds of deflation.

9. The method of claim 5, wherein the control system controls an inflation mode and a deflation mode of the at least one expandable member.

10. The method of claim 4, wherein the means for modulating the flow of fluid comprises a source of pressurized fluid in communication with the first lumen of the catheter, and wherein the control system is configured to modulate a flow of fluid exiting the first lumen of the catheter during time intervals when the at least one expandable member is in the blocking position.

11. The method of claim 10, wherein the flow of fluid through the first lumen of the catheter is controlled by the control system to maintain the flow of fluid at substantially a target pressure.

12. The method of claim 11, wherein the flow rate of fluid through the first lumen is increased over time to achieve the target pressure.

13. The method of claim 10, wherein the flow rate of fluid through the first lumen is a substantially constant flow.

14. The method of claim 10, wherein the flow rate of fluid through the first lumen is a modulated waveform flow.

15. The method of claim 10, wherein the flow rate of fluid is increased over time until a target flow rate is reached.

16. The method of claim 1, further comprising providing a means for delivering at least one drug, wherein the control system is configured to control the means for delivering at least one drug.

17. The method of claim 16, wherein the at least one drug comprises a plurality of drugs, and wherein the control system is configured to control the delivery of each respective drug of the plurality of drugs.

18. The method of claim 17, wherein the control system is configured to deliver the plurality of drugs substantially simultaneously.

19. The method of claim 17, wherein the control system is configured to deliver the plurality of drugs substantially sequentially.

20. The method of claim 17, wherein the control system is configured to deliver at least some of the plurality of drugs substantially simultaneously and at least some of the plurality of drugs substantially sequentially.

21. The method of claim 4, further comprising:
providing a pressurized supplied fluid in communication with the first lumen of the catheter; and
providing a means for regulating the temperature of the supplied fluid in communication with the first lumen of the catheter,
wherein the control system is in operable communication with the means for regulating the temperature of the supplied fluid, and wherein; upon activation, the control system modulates the temperature of the supplied fluid in communication with the first lumen of the catheter.

22. The method of claim 21, wherein the control system controls the means for regulating the temperature to maintain the temperature of the supplied fluid at the distal end of the first lumen substantially between about 18-37° C.

23. The method of claim 21, wherein the control system controls the means for regulating the temperature to decrease the temperature of the supplied fluid at the distal end of the first lumen toward a target temperature range of between about 18-37° C. over a time period.

24. The method of claim 23, wherein the control system controls the means for regulating the temperature to increase the temperature of the supplied fluid from the target temperature range.

25. A system for preventing injury to an organ or tissue in a subject during or after reperfusion following an ischemic event to the organ or tissue, comprising:
a catheter configured for insertion therein a vessel of the subject, wherein the catheter comprises a first lumen in communication with a distal end of the catheter and a distal region comprising at least one expandable member that is movable from and between a non-inflated position, in which fluid can pass the at least one expandable member, and a blocking position, in which the at least one expandable member is inflated to occlude the vessel such that fluid is prevented from passing by the at least one expandable member;
a means for modulating the flow of fluid within the vessel of the subject; and
a control system configured to control pulsation of the flow of fluid within the vessel for a desired period of time and at a desired pulsatile frequency, wherein the control system controls the means for modulating the flow of fluid.

26. The system of claim 25, wherein the control system is configured to be manually controlled or automatically controlled.

27. The system of claim 26, wherein the control system comprises at least one control algorithm that is adapted to automatically control the means for modulating the flow of fluid.

28. The system of claim 25, wherein the means for modulating the flow of fluid comprises the at least one expandable member, wherein the control system is configured to control an inflation mode and a deflation mode of the at least one expandable member to control the pulsatile flow of fluid past the at least one expandable member of the catheter.

29. The system of claim 28, wherein the control system is configured to control the pressure of the at least one expandable member during the inflation mode such that a target pressure is achieved therein the at least one expandable member.

30. The system of claim 28, wherein the control system controls the frequency of the cycle of movement of the at least one expandable member between the non-inflated position and the blocking position to obtain the desired pulsatile frequency.

31. The system of claim 30, wherein the frequency of the cycle of movement comprises successive periods of n seconds of inflation and m seconds of deflation.

32. The system of claim 26, wherein the means for modulating the flow of fluid comprises a source of pressurized fluid in communication with the first lumen of the catheter, wherein the control system is configured to modulate a flow of a fluid exiting the first lumen of the catheter to control the pulsatile flow of fluid within the vessel during time intervals when the at least one expandable member is in the blocking position.

33. The system of claim 32, further comprising a first pressure sensor mounted to a distal end portion of the catheter and a second pressure sensor positioned within a vessel of the subject remote from the catheter; wherein the first and second pressure sensors are in communication with the control system; and wherein the control system is configured to compare the pressures sensed at the respective first and second pressure sensors to a target pressure to derive a comparative pressure result and to control the flow of the fluid exiting the first lumen in response to the comparative pressure result.

34. The system of claim 32, further comprising a first pressure sensor mounted to a distal end portion of the catheter and in communication with the control system; and wherein the control system is configured to compare the pressure sensed at the first pressure sensor to a user defined target pressure to derive a comparative pressure result and to control the flow of the fluid exiting the first lumen in response to the comparative pressure result.

35. The system of claim 25, further comprising a means for delivering at least one drug to the subject, wherein the control system is configured to control the delivery of the at least one drug.

36. The system of claim 35, wherein the at least one drug comprises a plurality of drugs, and wherein the control system is configured to control the delivery of each respective drug of the plurality of drugs.

37. The system of claim 36, wherein the control system is configured to deliver the plurality of drugs substantially simultaneously.

38. The system of claim 36, wherein the control system is configured to deliver the plurality of drugs substantially sequentially.

39. The system of claim 36, wherein the control system is configured to deliver at least some of the plurality of drugs simultaneously and at least some of the plurality of drugs sequentially.

40. The system of claim 25, further comprising a means for regulating the temperature of a pressurized supplied fluid in communication with the first lumen of the catheter, wherein the control system is configured to control the means for regulating the temperature of the supplied fluid such that the temperature of the fluid being supplied to the first lumen is at a desired temperature.

41. The system of claim 40, wherein the control system controls the means for regulating the temperature of the supplied fluid such that the supplied fluid at the distal end of the first lumen is held substantially constant between about 18-37° C.

42. The system of claim 40, wherein the control system controls the means for regulating the temperature to decrease the temperature of the supplied fluid at the distal end of the first lumen toward a target temperature range of between about 18-37° C. over a time period.

43. The system of claim 42, wherein the control system controls the means for regulating the temperature of the supplied fluid to increase the temperature of the supplied fluid from the target temperature range over time.
